(19) United States
(12) Patent Application Publication
(10) Pub. No.: US 2005/0148616 A1
(43) Pub. Date: Jul. 7, 2005

(54) Inhibitor of TNF-alpha and IL-1beta production

(76) Inventors: Xiaoqiang Yan, Shanghai (CN); Zhimin Ma, Suzhou (CN); Tao Wang, Shanghai (CN)

Correspondence Address: FISH & RICHARDSON P.C., 225 Franklin Street, Boston, MA, 02110-2804, US

(21) Appl. No.: 11/004,142

(22) Filed: Dec. 3, 2004

Related U.S. Application Data
(60) Provisional application No. 60/526,873, filed on Dec. 3, 2003.

(51) Int. Cl. A61K 31/4745

(52) U.S. Cl. 514/284

(57) ABSTRACT

A method of inhibiting TNF-α or IL-1β expression with a compound of the following formula: 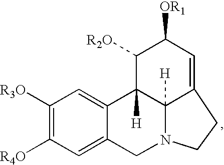
wherein each of R₁, R₂, R₃, and R₄, independently, is H, alkyl, aryl, or alkylcarbonyl, or R₁ and R₂ together, or R₃ and R₄ together are —(CH₂)ₙ—, n being 1, 2, or 3. Also disclosed is a method of treating a TNF-α or IL-1β related disorder using such a compound.

### RELATED APPLICATION

Pursuant to 35 U.S.C. § 119(e), this application claims the benefit of prior U.S. provisional application 60/526,873, filed Dec. 3, 2003.

### BACKGROUND

Tumor Necrosis Factor alpha (TNF-α), a mononuclear cytokine, is predominantly produced by monocytes and macrophages. It possesses various biological activities: (1) killing cancer cells or inhibiting growth of cancer cells, (2) enhancing phagocytosis of neutrophilic granulocyte, (3) killing infectious pathogens, and (4) increasing expression of adhesion molecules on vascular endothelial cells during inflammatory responses. Disorders related to expression of TNF-α include, but are not limited to, rheumatoid arthritis, juvenile rheumatoid arthritis, osteoarthritis, spondyloarthropathies, inflammatory bowel disease (including Crohn's disease and ulcerative colitis), chronic heart failure, systemic lupus erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, cancer (e.g., lung carcinomas, esophagus carcinoma, gastric adenocarcinoma, and prostate carcinoma), viral respiratory disease, and obesity. See, e.g., Ogata H. et al Curr Pharm Des. 2003; 9(14): 1107-13; Moller D. R. et al J Intern Med. 2003; 253(1): 31-40; Taylor P. C. et al Curr Pharm Des. 2003; 9(14): 1095-106; Wilkinson N. et al Arch Dis Child. 2003; 88(3): 186-91; Nishimura F. et al J Periodontol. 2003; 74(1): 97-102; Weinberg J. M. et al Cutis. 2003; 71(1): 41-5; Burnham E. et al Crit Care Med. 2001; 29(3): 690-1; Sack M. et al Pharmacol Ther. 2002; 94(1-2): 123-35; Barnes P. J. et al Annu Rev Pharmacol Toxicol. 2002; 42:81-98; Mageed R. A. et al Lupus 2002; 11 (12): 850-5; Tsimberidou A. M. et al Expert Rev Anticancer Ther. 2002; 2(3): 277-86; Muller T. et al Curr Opin Investig Drugs. 2002; 3(12): 1763-7; Calandra T. et al Curr Clin Top Infect Dis. 2002; 22:1-23; Girolomoni G et al Curr Opin Investig Drugs. 2002; 3(11): 1590-5; Tutuncu Z. et al Clin Exp Rheumatol. 2002; 20(6 Suppl 28): S146-51; Braun J. et al Best Pract Res Clin Rheumatol. 2002; 16(4): 631-51; Barnes P. J. et al Novartis Found Symp. 2001; 234:255-67; discussion 267-72; Brady M. et al Baillieres Best Pract Res Clin Gastroenterol. 1999; 13(2): 265-89; Goldring M. B. et al Expert Opin Biol Ther. 2001; 1(5): 817-29; Mariette X. Rev Prat. 2003; 53(5): 507-11; Sharma R. et al Int J Cardiol. 2002; 85(1): 161-71; Wang C. X. et al Prog Neurobiol. 2002; 67(2): 161-72; Van Reeth K. et al Vet Immunol Immunopathol. 2002; 87(3-4): 161-8; Leonard B. E. et al Int J Dev Neurosci. 2001; 19(3): 305-12; and Hays S. J. et al Curr Pharm Des. 1998; 4(4): 335-48.

Interleukin-1 beta (IL-1β), a cytokine secreted by cells such as monocytes, macrophages and dendritic cells, mediates a wide range of immune and inflammatory responses. One can modulate IL-1β production to treat a variety of disorders, such as rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, and scleroderma. See, e.g., Taylor P. C. et al Curr Pharm Des. 2003; 9(14): 1095-106; Dellinger R. P. et al Clin Infect Dis. 2003; 36(10): 1259-65; Takashiba S. et al J Periodontol. 2003; 74(1): 103-10; Diwan A. et al Curr Mol Med. 2003; 3(2): 161-82; Lundberg I. E. et al Rheum Dis Clin North Am. 2002; 28(4): 799-822; Makhija R. et al J Hepatobiliary Pancreat Surg. 2002; 9(4): 401-10; Chung K. F. et al Eur Respir J Suppl. 2001; 34:50s-59s; Hallegua D. S. et al Ann Rheum Dis. 2002; 61(11): 960-7; Goldring M. B. et al Expert Opin Biol Ther. 2001; 1(5): 817-29; Mrak R. E. et al Neurobiol Aging. 2001; 22(6): 903-8; Brady M. et al Baillieres Best Pract Res Clin Gastroenterol. 1999; 13(2): 265-89; Van der Meer J. W. et al Ann N Y Acad Sci. 1998; 856:243-51; Rameshwar P. et al Acta Haematol. 2003; 109(1): 1-10; de Kozak Y et al Int Rev Immunol. 2002; 21(2-3): 231-53; Wang C. X. et al Prog Neurobiol. 2002; 67(2): 161-72; Van Reeth K. et al Vet Immunol Immunopathol. 2002; 87(3-4): 161-8; Stirling R. G et al Br Med Bull. 2000; 56(4): 1037-53; Leonard B. E. et al Int J Dev Neurosci. 2001; 19(3): 305-12; Allan S. M. et al Ann N Y Acad Sci. 2000; 917:84-93; and Cafagna D. et al Minerva Med. 1998; 89(5): 153-61.

### SUMMARY

This invention is based on a surprising discovery that pseudolycorine inhibits expression of both TNF-α and IL-1β.

Thus, an aspect of this invention relates to a method of inhibiting expression of TNF-α or IL-1β in a subject in need thereof. The method includes administering to the subject an effective amount of a compound of the following formula: 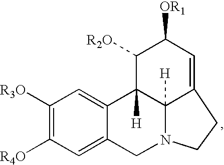
wherein each of R₁, R₂, R₃, and R_{4,} independently, is H, alkyl, aryl, or alkylcarbonyl, or R₁ and R₂ together, or R₃ and R₄ together are —(CH₂)ₙ—, n being 1, 2, or 3.

The term “alkyl” refers to a straight or branched hydrocarbon, containing 1-6 carbon atoms. Examples of alkyl groups include, but are not limited to, methyl, mehylene, ethyl, ethylene, n-propyl, i-propyl, n-butyl, i-butyl, and t-butyl.

The term “aryl” refers to a 6-carbon monocyclic or 10-carbon bicyclic aromatic ring system wherein each ring may have 1 to 4 substituents. Examples of aryl groups include phenyl, naphthyl, and the like.

The term “alkylcarbonyl” refers to carbonyl connected to a C₁₋₅ alkyl group by single bonding. Examples of alkylcarbonyl groups include, but are not limited to, acetyl, ethylcarbonyl, and n-propylcarbonyl, and i-propylcarbonyl.

The above-described compounds each contain an amino group and several chiral centers, and may have one or more phenylhydroxyl groups. Thus, they can occur as salts or isomeric forms. The salts include inorganic and organic acid addition salts such as hydrochloride, hydrobromide, phosphate, sulphate, citrate, lactate, tartrate, maleate, fumarate, mandelate and oxalate; and inorganic and organic base addition salts with bases such as sodium hydroxide, tris(hydroxymethyl) aminomethane, and N-methyl-glucamine. Isomeric forms include racemates and racemic mixtures, single enantiomers, individual diastereomers, and diastereomeric mixtures. All such salts and isomers are contemplated. The compounds can also occur as produrgs. For general discussion of produgs, see, e.g., Albert S. Kearney Advanced Drug Reviews. 19: 229-234 (1996).

Shown below are two examples of the above-described compounds, i.e., pseudolycorine and pseudolycorine hydrochloride:

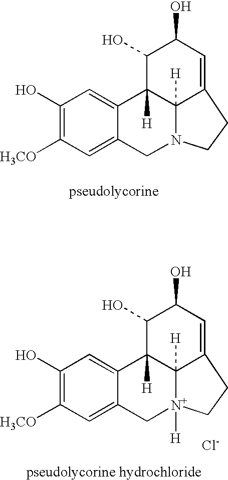

Another aspect of this invention relates to a method for treating a TNF-α related disorder or an IL-1β related disorder including administering to a subject in need thereof an effective amount of one of the above-described compounds.

A TNF-α related disorder and an IL-1β related disorder may be induced by over-expression of TNF-α and of IL-1β, respectively. Examples of TNF-α related disorders include, but are not limited to, rheumatoid arthritis, juvenile rheumatoid arthritis, osteoarthritis, spondyloarthropathies, inflammatory bowel disease, chronic heart failure, diabetes mellitus, systemic lupus, erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, lung carcinomas, esophagus carcinoma, gastric adenocarcinoma, prostate carcinoma, viral respiratory disease, and obesity. Examples of IL-1β related disorders include, but are not limited to, rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, and scleroderma.

Also within the scope of this invention is a pharmaceutical composition containing an effective amount of one of the above-described compounds and a pharmaceutically acceptable carrier, as well as the use of such a composition for the manufacture of a medicament for treating a TNF-α related disorder or an IL-1β related disorder.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows inhibitory effect of pseudolycorine hydrochloride on lipopolysaccharide (LPS)-induced TNF-α production in human normal peripheral blood monocytes (PBMCs).

FIG. 2 shows inhibitory effect of pseudolycorine hydrochloride on LPS-induced IL-1β production in human normal PBMCs.

FIG. 3 shows concentration-dependent inhibitory effect of pseudolycorine hydrochloride on LPS-induced TNF-α production.

FIG. 4 shows concentration-dependent inhibition effect of pseudolycorine hydrochloride on LPS-induced IL-1β production.

### DETAILED DESCRIPTION

This invention includes methods of inhibiting expression of TNF-α or IL-1β, treating a TNF-α related disorder, and treating an IL-1β related disorder with an effective amount of one of the above-described compounds. The term “an effective amount” refers to the amount of the compound that is required to confer therapeutic effect in a subject. Effective amounts may vary, as recognized by those skilled in the art, depending on route of administration, excipient usage, and optional co-usage with another therapeutic agent. The term “treating” refers to administering a compound to a subject that has a TNF-α related disorder or an IL-1β related disorder, or has a symptom of the disorder, or has a predisposition toward the disorder, with the purpose to cure, heal, alleviate, relieve, alter, remedy, ameliorate, improve, or affect the disorder, the symptoms of the disorder, or the predisposition toward the disorder.

Some of the compounds used in the above-described methods are naturally occurring. For example, pseudolycorine can be isolated from bulbs of Narcissus tazetta, a medicinal plant, according to the procedure described in Furusawa E. et al., Pro Soc Expt Biol & Med 1971; 136:1168. Others can be prepared by modification of naturally occurring compounds or by total synthesis, using chemical transformations and protecting group methodologies (protection and deprotection) well known in the art. See, e.g., Larock R. Comprehensive Organic Transformations, VCH Publishers, 1989; T. W. Greene and Wuts P. G. M. Protective Groups in Organic Synthesis, 3^{rd} Ed., John Wiley and Sons, 1999; Fieser L. and Fieser M., Fieser and Fieser's Reagents for Organic Synthesis, John Wiley and Sons, 1994; and Paquette L. ed., Encyclopedia of Reagents for Organic Synthesis, John Wiley and Sons, 1995 and subsequent editions thereof. As an example, pseudolycorine hydrochloride, a salt of pseudolycorine, can be prepared by treating pseudolycorine with hydrogen chloride.

To practice one of the above-described methods, one administers to a subject in need thereof orally, rectally, parenterally, by inhalation spray, or via an implanted reservoir a composition that is either one of the above-described compounds alone or a mixture of the compound and a pharmaceutically acceptable carrier. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (e.g., containing more than 12 carbon atoms). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense of being compatible with the active ingredient of the formulation (and preferably, capable of stabilizing it) and not deleterious to the subject to be treated. For example, solubilizing agents, such as cyclodextrins can be utilized as pharmaceutical excipients for delivery of the active compounds. Examples of other carriers include colloidal silicon dioxide, magnesium stearate, cellulose, sodium lauryl sulfate, and D&C Yellow # 10.

A suitable in vitro assay can be used to preliminarily evaluate the efficacy of one of the above-described compounds in inhibiting expression of TNF-α or IL-1β expression. The compound can further be examined for its efficacy in treating a TNF-α related disorder or an IL-1β related disorder by in vivo assays. For example, it can be administered to an animal (e.g., a mouse model) having a TNF-α or IL-1β related disorder and its therapeutic effect is then assessed. Based on the results, an appropriate dosage range and administration route can also be determined.

Without further elaboration, it is believed that the above description has adequately enabled the present invention. The following specific examples are, therefore, to be construed as merely illustrative, and not limitative of the remainder of the disclosure in any way whatsoever. All of the publications, including U.S. provisional application 60/526,873, cited herein are hereby incorporated by reference in their entirety.

Example: Inhibition of the Expression of TNF-α and IL-1β

PBMCs were isolated from fresh blood using a Ficoll-Paque Plus reagent (Amersham Bioscience) according to the protocol recommended by the manufacturer. The cells were suspended in a RPMI 1640 medium containing 10% FBS at a concentration of 1×10⁵ cells/ml and seeded in a 96-well plate (1×10⁴ cells total in each well). Each reaction was carried out in three wells.

10 μl of pseudolycorine hydrochloride in DMSO was added to wells to obtain the final concentrations at 0.1, 0.3, 1, 3, 10, 30, and 100 μg/ml. In a positive control, dexamethason (DEX), an anti-inflammatory agent, instead of pseudolycorine hydrochloride, was added (final concentration 10 μM). In a negative control, 10 μl of the medium, instead of pseudolycorine hydrochloride, was added. The plate was placed in a 5% CO₂ incubator at 37° C. for 15 minutes. After 10 μl aliquots of 100 μg/ml of LPS were added to all wells except for the negative control, the plate was placed in a 5% CO₂ incubator at 37° C. overnight.

The plate was spun at 1000 rpm for 15 minutes and the supernatants were collected. The concentrations of TNF-α and IL-1β were measured by the TNF-α ELISA (Enzyme Linked Immunosorbent Assay) Kit and IL-1β ELISA Kit (Jingmei Bioengineer Technology). The inhibition ratios of TNF-α and IL-1β were calculated as follows:
TNF-α Inhibition Ratio (%)=[TNF-α]_{control}−[TNF-α]_{PHCl}/[TNF-α]_{control}×100%
IL-1β Inhibition Ratio (%)=[IL-1β]_{control}−[IL-1β]_{PHCl}/[IL-1β]_{control}×100%
where [TNF-α]_{PHCl} is the concentration of TNF-α in PBMCs treated with pseudolycorine hydrochloride and LPS, [TNF-α]_{control} is the concentration of TNF-α in PBMCs treated with LPS and the medium, [IL-1β]_{PHCl} is the concentration of IL-1β in PBMCs treated with pseudolycorine hydrochloride and LPS, and [IL-1β]_{control} is the concentration of IL-1β in PBMCs treated with LPS and the medium.

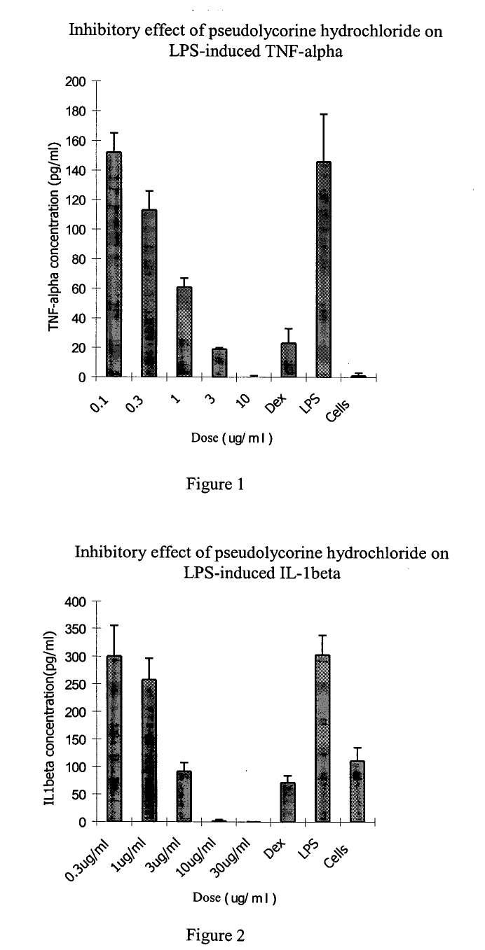

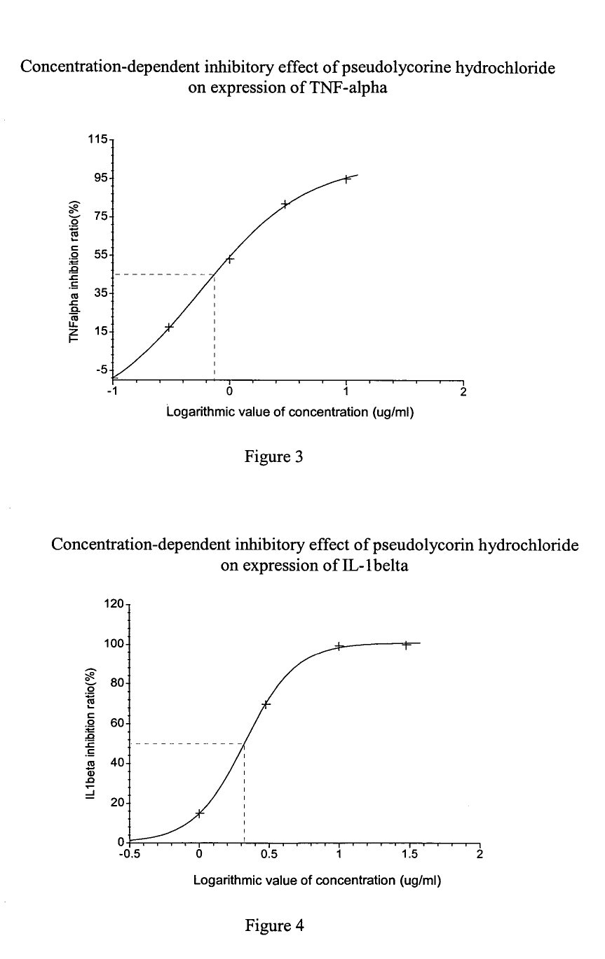

The results show that pseudolycorine hydrochloride, unexpectedly, inhibited expression of TNF-α when its concentration was equal to or higher than 0.3 μg/ml (FIG. 1), and expression of IL-1β when its concentration is equal to or higher than 1 μg/ml (FIG. 2). Also unexpectedly, pseudolycorine hydrochloride at a concentration of 3 μg/ml or higher inhibited expression of TNF-α more effectively than DEX at a concentration of 10 μM, and pseudolycorine hydrochloride at a concentration of 10 μg/ml or higher inhibited expression of IL-1β more effectively than DEX at a concentration of 10 μM. FIGS. 3 and 4 show that pseudolycorine hydrochloride inhibited expression of both TNF-α and IL-1β in a concentration-dependent manner.

### OTHER EMBODIMENTS

A number of embodiments of the invention have been described. Nevertheless, it will be understood that various modifications may be made without departing from the spirit and scope of the invention. Accordingly, other embodiments are also within the scope of the following claims.

The details of one or more embodiments of the invention are set forth in the description below. Other features, objects, and advantages of the invention will be apparent from the description and from the claims.

1. A method of inhibiting the expression of TNF-α in a subject in need thereof, comprising administering to the subject an effective amount of a compound of the following formula: 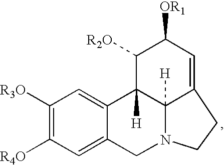
wherein each of R₁, R₂, R₃, and R_{4,} independently, is H, alkyl, aryl, or alkylcarbonyl, or R and R₂ together, or R₃ and R₄ together are —(CH₂)ₙ—, n being 1, 2, or 3.

2. The method of claim 1, wherein each of R₁ and R₂ is H.

3. The method of claim 2, wherein one of R₃ and R₄ is H.

4. The method of claim 3, wherein R₃ is H and R₄ is alkyl.

5. The method of claim 4, wherein R₄ is methyl.

6. A method of inhibiting the expression of IL-1β in a subject in need thereof, comprising administering to the subject an effective amount of a compound of the following formula: 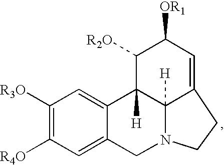
wherein each of R₁, R₂, R₃, and R_{4,} independently, is H, alkyl, aryl, or alkylcarbonyl, or R₁ and R₂ together, or R₃ and R₄ together are —(CH₂)ₙ—, n being 1, 2, or 3.

7. The method of claim 6, wherein each of R₁ and R₂ is H.

8. The method of claim 7, wherein one of R₃ and R₄ is H.

9. The method of claim 8, wherein R₃ is H and R₄ is alkyl.

10. The method of claim 9, wherein R₄ is methyl.

11. A method for treating a TNF-α related disorder, comprising administering to a subject in need thereof an effective amount of the compound of the following formula: 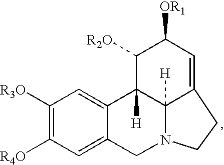
wherein each of R₁, R₂, R₃, and R_{4,} independently, is H, alkyl, aryl, or alkylcarbonyl, or R₁ and R₂ together, or R₃ and R₄ together are —(CH₂)ₙ—, n being 1, 2, or 3; the TNF-α related disorder being rheumatoid arthritis, juvenile rheumatoid arthritis, osteoarthritis, spondyloarthropathies, inflammatory bowel disease, chronic heart failure, diabetes mellitus, systemic lupus, erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, viral respiratory disease, or obesity.

12. The method of claim 11, wherein each of R₁ and R₂ is H.

13. The method of claim 12, wherein one of R₃ and R₄ is H.

14. The method of claim 13, wherein R₃ is H and R₄ is alkyl.

15. The method of claim 14, wherein R₄ is methyl.

16. The method of claim 15, wherein the TNF-α related disorder is rheumatoid arthritis, juvenile rheumatoid arthritis, osteoarthritis, inflammatory bowel disease, systemic lupus, psoriasis, sepsis, or septic shock.

17. A method for treating an IL-1β related disorder, comprising administering to a subject in need thereof an effective amount of a compound of the following formula: 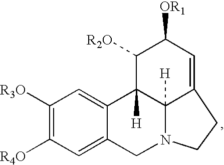
wherein each of R₁, R₂, R₃, and R₄, independently, is H, alkyl, aryl, or alkylcarbonyl, or R₁ and R₂ together, or R₃ and R₄ together are —(CH₂)ₙ—, n being 1, 2, or 3; the IL-1β related disorder being rheumatoid arthritis, hematosepsis, periodontal disease, chronic heart failure, polymyositis/dermatomyositis, acute pancreatitis, chronic obstructive pulmonary disease, Alzheimer's disease, osteoarthritis, bacterial infections, multiple myeloma, myelodysplastic syndrome, uveitis, central nervous system injury, viral respiratory disease, asthma, depression, or scleroderma.

18. The method of claim 17, wherein each of R₁ and R₂ is H.

19. The method of claim 18, wherein one of R₃ and R₄ is H.

20. The method of claim 19, wherein R₃ is H and R₄ is alkyl.

21. The method of claim 20, wherein R₄ is methyl.

22. The method of claim 21, wherein the IL-1β related disorder is rheumatoid arthritis, or osteoarthritis.

23. A method for treating cancer, comprising administering to a subject in need thereof an effective amount of a compound of the following formula: 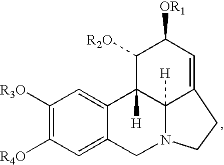
wherein each of R₁, R₂, R₃, and R₄, independently, is H, alkyl, aryl, or alkylcarbonyl, or R₁ and R₂ together, or R₃ and R₄ together are —(CH₂)ₙ—, n being 1, 2, or 3; the cancer being lung carcinomas, esophagus carcinoma, gastric adenocarcinoma, or prostate carcinoma.

24. The method of claim 23, wherein each of R₁ and R₂ is H.

25. The method of claim 24, wherein one of R₃ and R₄ is H.

26. The method of claim 25, wherein R₃ is H and R₄ is alkyl.

27. The method of claim 26, wherein R₄ is methyl.
